(19) United States
(12) Patent Application Publication
(10) Pub. No.: US 2012/0123317 A1
(43) Pub. Date: May 17, 2012

(54) METHODS FOR IMPLANATION OF GLAUCOMA SHUNTS

(75) Inventors: Christopher Horvath, Desert Hot Springs, CA (US); Laszlo O. Romoda, San Clemente, CA (US)

(73) Assignee: AQUESYS, INC., Irvine, CA (US)

(21) Appl. No.: 12/946,556

(22) Filed: Nov. 15, 2010

(51) Int. Cl. A61M 5/00

(52) U.S. Cl. 604/8

(57) ABSTRACT

The present invention generally relates to the reduction of intraocular pressure, and in particular, to improved methods for implanting an intraocular shunt in the eye to treat glaucoma. The methods provide for implantation of an intraocular shunt while minimizing the risk of severe eye trauma due to the interaction between the deployment device and the surrounding eye tissue.

### FIELD OF THE INVENTION

The present invention generally relates to methods for implanting intraocular shunts into the eye while avoiding or minimizing damage to the surrounding eye tissue during or after deployment of a shunt into the eye.

### BACKGROUND

Glaucoma is a disease in which the optic nerve is damaged, leading to progressive, irreversible loss of vision. It is typically associated with increased pressure of the fluid (i.e., aqueous humor) in the eye. Untreated glaucoma leads to permanent damage of the optic nerve and resultant visual field loss, which can progress to blindness. Once lost, this damaged visual field cannot be recovered. Glaucoma affects 1 in 200 people aged fifty and younger, and 1 in 10 over the age of eighty for a total of approximately 70 million people worldwide, and glaucoma is the second leading cause of blindness in the world.

The importance of lowering intraocular pressure (IOP) in delaying glaucomatous progression has been well documented. Various surgical filtration methods for lowering IOP have been described. One method involves entering the eye though the conjunctiva and inwards through the sclera (i.e., an ab externo procedure) to reach a drainage structure such as Schlemm's canal. Another method involves inserting a shunt into the eye through the cornea, across the anterior chamber, and through the trabecular meshwork and sclera (i.e., an ab interno approach) to create a fluid flow path between the anterior chamber of the eye and a region of lower pressure in the eye such as Schlemm's canal, the episcleral vein, the suprachoroidal space, or the subconjunctival space. Such fluid flow pathways allow for aqueous humor to exit the anterior chamber, thereby reducing IOP.

Various manual and automated deployment devices for implanting an intraocular shunt have been described. See, for example, U.S. Pat. No. 6,544,249 and U.S. patent application publication number 2008/0108933. Most deployment devices are coupled to a hollow needle which holds the intraocular shunt. Whether an ab externo approach or an ab interno approach is used, the needle is inserted into the eye to deploy the intraocular shunt into the eye. The needle is then withdrawn from the eye. Complications can arise with such shunt implantation methods.

### SUMMARY

The invention relates to eliminating or at least minimizing damage to the eye of a patient during an intraocular shunt placement procedure. Intraocular shunts are typically deployed into the eye using a deployment device that includes or is coupled to a hollow shaft, such as a needle, that holds the intraocular shunt. The hollow shaft of the deployment device is inserted into the eye, then the shunt is deployed into the eye from the deployment device. Once inserted into the eye, the interaction between the hollow shaft of the deployment device and surrounding eye tissue oftentimes causes the shaft to become stuck in the surrounding eye tissue (due to frictional resistance, for example), which can cause severe eye trauma upon shunt deployment or withdrawal of the shaft from the eye. This trauma is avoided or at least minimized according to the invention by loosening the hollow shaft from the surrounding eye tissue prior to deploying the shunt into the eye from the deployment device and/or withdrawing the hollow shaft from the eye.

The present invention provides improved methods for implantation of intraocular shunts. In one aspect, the methods of the invention involve the insertion into the eye of a portion of a deployment device comprising an intraocular shunt, loosening the portion of deployment device from the surrounding eye tissue, deploying the shunt into the eye from the deployment device, then withdrawing the portion of the deployment device from the eye. In one particular embodiment, the methods involve inserting into the eye a portion of a deployment device comprising an intraocular shunt without removing an anatomical feature of the eye, loosening the portion of the deployment device from the surrounding eye tissue, deploying the shunt into the eye from the deployment device, then withdrawing the portion of the deployment device from the eye. Loosening of the portion of the deployment device inserted into the eye from the surrounding eye tissue can be achieved, for example, by rotating the deployment device or a portion of the deployment device, other than the portion inserted into the eye. Rotation of the deployment device, or portion thereof, causes the portion of the deployment device inserted into the eye to also rotate, thereby loosening the deployment device from the surrounding eye tissue. Examples of eye tissue surrounding the portion of the deployment device inserted into the eye include, without limitation, the scleral tissue and/or the trabecular meshwork.

The loosening and deployment steps of the methods of the invention do not have to be conducted in any particular order. For example, the methods of the invention may involve inserting into the eye a portion of a deployment device comprising an intraocular shunt, deploying the shunt into the eye from the deployment device, loosening the portion of the deployment device from the surrounding eye tissue, then withdrawing the portion of the deployment device from the eye.

The deployment device may be configured such that a proximal portion of the deployment device is rotated to loosen the portion of the deployment device in the eye from the surrounding eye tissue before or after deploying the shunt into the eye. Alternatively, the deployment device may be configured such that a distal portion of the deployment device is rotated to loosen the portion of the deployment device in the eye from the surrounding eye before or after deploying the shunt into the eye. In yet another embodiment, the entire deployment device may be rotated to loosen the portion of the deployment device in the eye from the surrounding eye tissue before or after deploying the shunt into the eye. Preferably, the deployment device, or a portion thereof, is rotated about its longitudinal axis. Rotation can be in a clockwise or counterclockwise direction.

In another aspect, the present invention relates to methods for implanting an intraocular shunt into an eye by inserting into the eye a portion of a deployment device comprising an intraocular shunt, whereby insertion into the eye is at an angle above or below the corneal limbus, rather than through the corneal limbus. Preferably, the portion of the deployment device is inserted into the eye at an angle above the corneal limbus. For example, a portion of a deployment device comprising an intraocular shunt is inserted into the eye approximately 1 mm to 2 mm above the corneal limbus, or any specific value within said range, e.g., 1 mm, 1.1 mm, 1.2 mm, 1.3 mm, 1.4 mm, 1.5 mm, 1.6 mm, 1.7 mm, 1.8 mm, 1.9 mm or 2 mm above the corneal limbus. The shunt is then deployed into the eye from the deployment device, and the portion of the deployment device is withdrawn from the eye. Shunt implantation methods above or below the corneal limbus are preferably coupled with the step of loosening the deployment device from the surrounding eye tissue before or after deploying the shunt into the eye, as previously described.

The methods of the invention are preferably conducted without removing an anatomical feature of the eye, such as the trabecular meshwork, the iris, the cornea, or the aqueous humor. In certain embodiments, the methods of the invention are conducted without inducing substantial ocular inflammation such as, for example, subconjunctival blebbing or endophthalmitis. Preferably, the methods of the invention are conducted using an ab interno approach by inserting a portion of a deployment device comprising an intraocular shunt through the cornea, across the anterior chamber, through the sclera and into an aqueous humor drainage structure such as the intra-Tenon's space, the subconjunctival space, the episcleral vein the suprachoroidal space or Schlemm's canal. Such an approach is contrasted with an ab externo approach which involves inserting the portion of the deployment device comprising an intraocular shunt from the outside of the eye through the conjunctiva and inward through the sclera to reach a drainage structure such as Schlemm's canal. Although, methods of the invention may be conducted using an ab externo approach.

In other certain embodiments, the methods of the invention are conducted without the use of an optical apparatus, particularly an optical apparatus that directly contacts the eye, such as a goniolens. In yet other certain embodiments, the methods of the invention are conducted using an optical apparatus that does not directly contact the eye, such as an ophthalmic microscope.

In a particular embodiment, the methods of the invention are reversible. That is, intraocular shunts that are implanted into the eye in accordance with the methods of the invention can be removed from the eye and a second shunt can be implanted in the eye.

Deployment of an intraocular shunt into the eye in accordance with the methods of the invention results in the formation of a passage that directs aqueous humor fluid flow from an area of high pressure in the eye, typically the anterior chamber, to an area of lower pressure within the eye, such as the intra-Tenon's space, the subconjunctival space, the episcleral vein, the suprachoroidal space or Schlemm's canal. Alternatively, the shunt is deployed in accordance with the methods of the invention such that it form a passage that directs aqueous humor fluid flow from an area of high pressure, such as the anterior chamber, to an area of lower pressure within the head, such as the subarachnoid space. In a preferred embodiment, deployment of an intraocular shunt in accordance with the methods of the invention results in the formation of a passage that directs aqueous humor fluid flow from the anterior chamber of the eye to the intra-Tenon's space.

### BRIEF DESCRIPTION OF THE DRAWINGS

FIG. 1 provides a cross-sectional diagram of the general anatomy of the eye.

FIG. 2 provides another cross-sectional view the eye, and certain anatomical structures of the eye.

FIG. 3 depicts, implantation of an intraocular shunt with a distal end of a deployment device holding a shunt, shown in cross-section.

FIG. 4 depicts a deployment device having a plunger type mechanism for deploying an intraocular shunt into the eye.

FIG. 5 depicts an example of a deployment device configured to hold an intraocular shunt.

FIG. 6A depicts a hollow shaft having a bend in a distal portion of the shaft. FIG. 6B depicts a hollow shaft having a U-shape. FIG. 6C depicts a hollow shaft having a V-shape.

FIG. 7A depicts a simulation of the exit site distance from the limbus and height above the iris after needle entry at the limbus using an ab interno procedure. FIG. 7B depicts a simulation of the exit site distance from the limbus and height above the iris after needle entry above the limbus using an ab interno procedure.

FIG. 8 is a schematic showing an embodiment of a shunt deployment device according to the invention

FIG. 9 shows an exploded view of the device shown in FIG. 17.

FIGS. 10A to 10D are schematics showing different enlarged views of the deployment mechanism of the deployment device.

FIGS. 11A to 11C are schematics showing interaction of the deployment mechanism with a portion of the housing of the deployment device.

FIG. 12 shows a cross sectional view of the deployment mechanism of the deployment device.

FIGS. 13A and 13B show schematics of the deployment mechanism in a pre-deployment configuration. FIG. 13C shows an enlarged view of the distal portion of the deployment device of FIG. 13A. This figure shows an intraocular shunt loaded within a hollow shaft of the deployment device.

FIGS. 14A and 14B show schematics of the deployment mechanism at the end of the first stage of deployment of the shunt from the deployment device. FIG. 14C shows an enlarged view of the distal portion of the deployment device of FIG. 14A. This figure shows an intraocular shunt partially deployed from within a hollow shaft of the deployment device.

FIG. 15A shows a schematic of the deployment device after deployment of the shunt from the device. FIG. 15B show a schematic of the deployment mechanism at the end of the second stage of deployment of the shunt from the deployment device. FIG. 15C shows an enlarged view of the distal portion of the deployment device after retraction of the shaft with the pusher abutting the shunt. FIG. 15D shows an enlarged view of the distal portion of the deployment device after deployment of the shunt.

FIGS. 16 and 17 show an intraocular shunt deployed within the eye. A proximal portion of the shunt resides in the anterior chamber and a distal portion of the shunt resides within the intra-Tenon's space. A middle portion of the shunt resides in the sclera.

### DETAILED DESCRIPTION

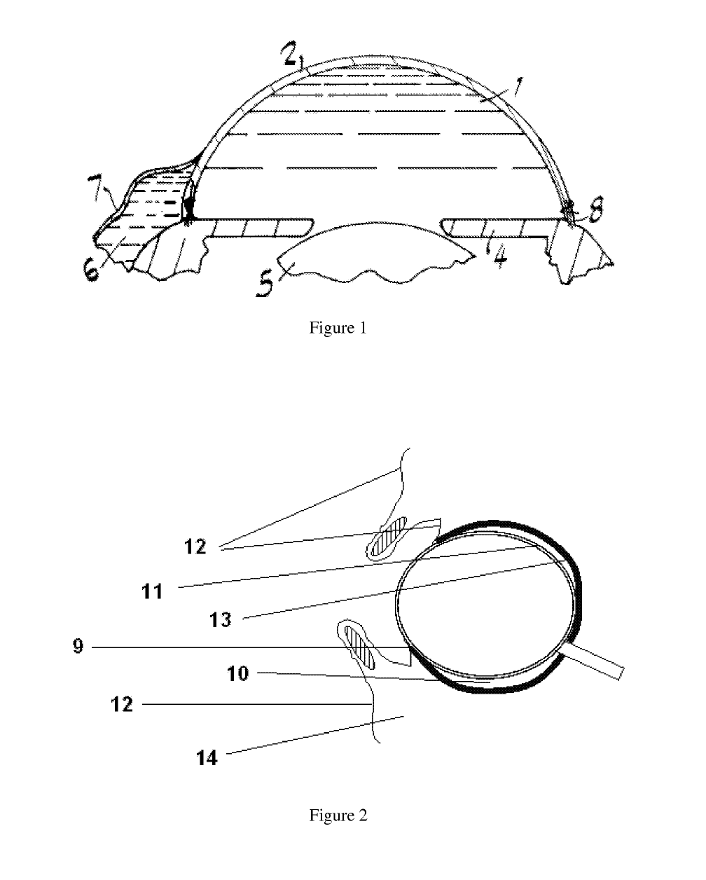

FIG. 1 provides a schematic diagram of the general anatomy of the eye. An anterior aspect of the anterior chamber 1 of the eye is the cornea 2, and a posterior aspect of the anterior chamber 1 of the eye is the iris 4. Beneath the iris 4 is the lens 5. The anterior chamber 1 is filled with aqueous humor 3. The aqueous humor 3 drains into a space(s) 6 below the conjunctiva 7 through the trabecular meshwork (not shown in detail) of the sclera 8. The aqueous humor is drained from the space(s) 6 below the conjunctiva 7 through a venous drainage system (not shown).

FIG. 2 provides a cross-sectional view of a portion of the eye, and provides greater detail regarding certain anatomical structures of the eye. In particular, FIG. 2 shows the relationship of the conjunctiva 12 and Tenon's capsule 13. Tenon's capsule 13 is a fascial layer of connective tissue surrounding the globe and extra-ocular muscles. As shown in FIG. 2, it is attached anteriorly to the limbus of the eye and extends posteriorly over the surface of the globe until it fuses with the dura surrounding the optic nerve. In FIG. 2, number 9 denotes the limbal fusion of the conjunctiva 12 and Tenon's capsule 13 to the sclera 11. The conjunctiva 12 and Tenon's capsule 13 are separate membranes that start at the limbal fusion 9 and connect to tissue at the posterior of the eye. The space formed below the conjunctiva 12 is referred to as the subconjunctival space, denoted as number 14. Below Tenon's capsule 13 there are Tenon's adhesions that connect the Tenon's capsule 13 to the sclera 11. The space between Tenon's capsule 13 and the sclera 11 where the Tenon's adhesions connect the Tenon's capsule 13 to the sclera 11 is referred to as the intra-Tenon's space, denoted as number 10.

In conditions of glaucoma, the pressure of the aqueous humor in the eye (anterior chamber) increases and this resultant increase of pressure can cause damage to the vascular system at the back of the eye and especially to the optic nerve. The treatment of glaucoma and other diseases that lead to elevated pressure in the anterior chamber involves relieving pressure within the anterior chamber to a normal level.

Glaucoma filtration surgery is a surgical procedure typically used to treat glaucoma. The procedure involves placing a shunt in the eye to relieve intraocular pressure by creating a pathway for draining aqueous humor from the anterior chamber of the eye. The shunt is typically positioned in the eye such that it creates a fluid-flow pathway between the anterior chamber of the eye and a region of lower pressure. Various structures and/or regions of the eye having lower pressure that have been targeted for aqueous humor drainage include Schlemm's canal, the subconjunctival space, the episcleral vein, the suprachoroidal space, or the subarachnoid space. Methods of implanting intraocular shunts are known in the art. Shunts may be implanted using an ab externo approach (entering through the conjunctiva and inwards through the sclera) or an ab interno approach (entering through the cornea, across the anterior chamber, and through the trabecular meshwork and sclera).

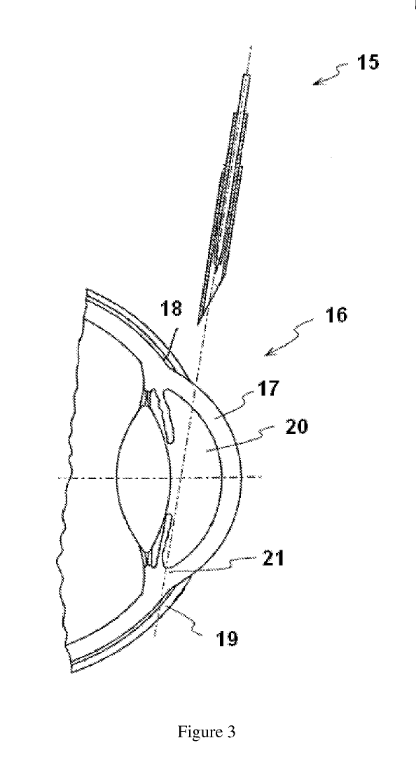

Ab interno approaches for implanting an intraocular shunts have been described and may vary depending on the structure targeted for aqueous humor drainage. For example, ab interno approaches for implanting an intraocular shunt into the subconjunctival space are shown in Yu et al. (U.S. Pat. No. 6,544,249 and U.S. patent publication number 2008/0108933) and Prywes (U.S. Pat. No. 6,007,511), the contents of each of which are incorporated by reference herein in its entirety. Briefly and with reference to FIG. 3, a surgical intervention to implant the shunt involves inserting into the eye a portion of a deployment device 15 that holds an intraocular shunt, and deploying the shunt within the eye 16. The portion of the deployment device 15 holding the shunt enters the eye 16 through the cornea 17 (ab interno approach). The portion of the deployment device 15 is advanced across the anterior chamber 20 (as depicted by the broken line) in what is referred to as a transpupil implant insertion. The portion of the deployment device 15 is advanced through the sclera 21 until a distal portion of the device is in proximity to the subconjunctival space. The shunt is then deployed from the deployment device, producing a conduit between the anterior chamber and the subconjunctival space to allow aqueous humor to drain through the conjunctival lymphatic system.

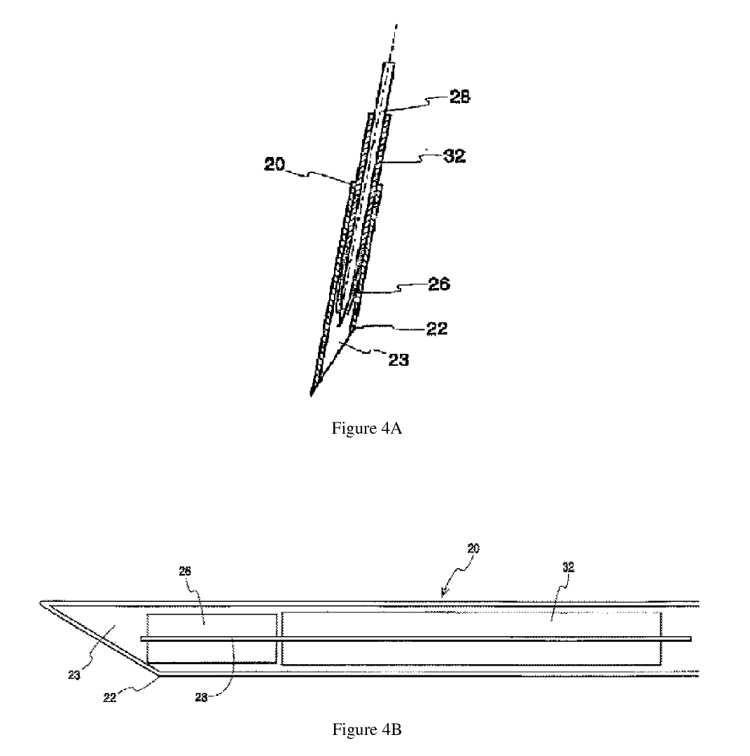

Previously proposed deployment devices for implanting an intraocular shunt into the eye, whether using an ab externo procedure or an ab interno procedure, typically include a plunger-type mechanism for deploying the shunt into the eye, such as the deployment device illustrated in FIG. 3. The deployment device in FIG. 3 is shown larger in FIG. 4A, and the distal portion of the deployment device is shown magnified in FIG. 4B. As shown in FIGS. 4A and 4B, the deployment device includes an assembly 20 that includes a hollow shaft 22 defining an inner chamber 23. Placed within the inner chamber 23 of the hollow shaft 22 is a cylindrical inner tube or plunger 32 that is coaxial with the shaft 22. In the loaded and ready to use condition, the intraocular shunt 26 is also placed or otherwise disposed within the hollow inner chamber 23 of the shaft 22 and is distally located relative to plunger 32. Both the intraocular shunt 26 and plunger 32 may be placed over and supported by optional guidewire 28. The intraocular shunt is deployed into the eye by advancing the plunger to push the intraocular shunt from the shaft into the eye. The shaft is then withdrawn from the eye.

However, complications can arise when using such deployment devices due to the frictional interaction between the deployment device and the surrounding eye tissue that results upon insertion of the deployment device into the eye and/or deployment of the intraocular shunt into the eye from the deployment device. Moderate to severe eye trauma can occur, beyond any trauma due to insertion of the deployment device, if the portion of the deployment device inserted into the eye is not loosened before or after deployment of the intraocular shunt from the device and prior to withdrawing the portion of the deployment device from the eye.

The present invention provides improved methods for implanting an intraocular shunt into the eye while avoiding or at least minimizing the amount of trauma to the eye that is typically involved with shunt implantation procedures. According to the methods of the invention, any frictional resistance between the deployment device and surrounding eye tissue that is created upon insertion of a portion of a deployment device in the eye is resolved by loosening the portion of the deployment device from the surrounding eye tissue before or after deployment of the intraocular shunt from the device and prior to withdrawing the portion of the deployment device from the eye. The methods can be used in conjunction with any known shunt deployment device, and in particular, any deployment device that includes a portion for holding an intraocular shunt or is coupled to a hollow shaft which is configured to hold an intraocular shunt.

Preferably, at least a portion of the deployment device is rotated before the shunt is deployed into the eye from the deployment device, in order to loosen the portion of the device inserted into the eye from the surrounding eye tissue prior to withdrawing the deployment device from the eye. Rotation may be clockwise or counterclockwise, and may be performed manually or in an automated manner. Rotation of only a distal portion of the deployment device may be sufficient to loosen the portion of the deployment device in the eye from the surrounding eye tissue, depending on the configuration of the device. Alternatively, rotation of the entire deployment device serves to loosen the portion of the deployment device in the eye from the surrounding eye tissue. Rotation of the deployment device, or a portion thereof, causes the portion of the deployment device that is inserted into the eye to also rotate, thereby loosening the portion of the deployment device in the eye form the surrounding eye tissue. Examples of surrounding eye tissue include but are not limited to the scleral tissue and the trabecular meshwork.

The deployment device, or a portion thereof, is rotated clockwise or counterclockwise about the longitudinal axis of the deployment device itself. The rotation about the longitudinal axis is preferably between 1° and 360°, or any specific value within said range, e.g., 1°, 3°, 5°, 10°, 15°, 30°, 45°, 60°, 75°, 90°, 105°, 120°, 135°, 150° 165°, 180°, 195°, 210°, 225°, 240°, 255°, 270°, 285°, 300°, 315°, 330°, 345° or 360°.

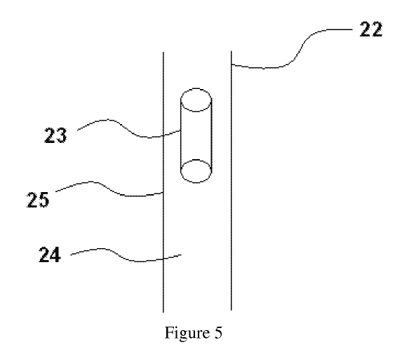

As previously stated, the methods of the invention can be used in conjunction with any shunt deployment device. FIG. 5 provides an exemplary schematic of a hollow shaft for use in conjunction with a deployment device in accordance with the methods of the invention. This shows hollow shaft 22 that is configured to hold an intraocular shunt 23. The shaft may hold the shunt within the hollow interior 24 of the shaft. Alternatively, the hollow shaft may hold the shunt on an outer surface 25 of the shaft. In particular embodiments, the shunt is held within the hollow interior of the shaft 24. Generally, in one embodiment, the intraocular shunts are of a cylindrical shape and have an outside cylindrical wall and a hollow interior. The shunt may have an inside diameter of approximately 10-250 microns, an outside diameter of approximately 190-300 microns, and a length of approximately 0.5 mm to 20 mm, such as, for example, 6 mm to 14 mm. The hollow shaft 22 is configured to at least hold a shunt of such shape and such dimensions. However, the hollow shaft 22 may be configured to hold shunts of different shapes and different dimensions than those described above, and the invention encompasses a shaft 22 that may be configured to hold any shaped or dimensioned intraocular shunt.

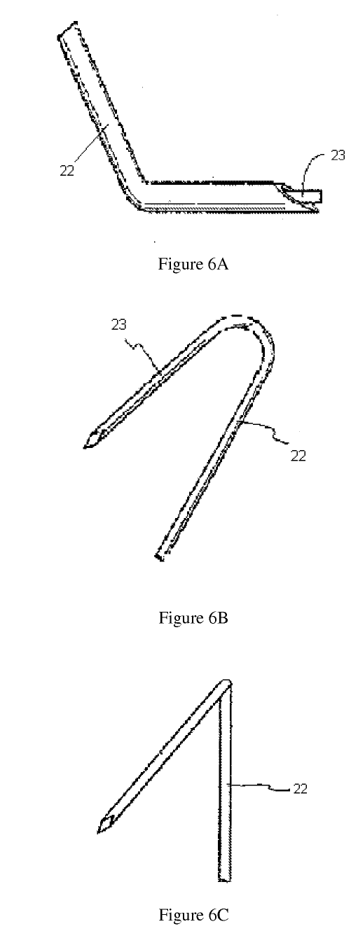

In some embodiments, the hollow shaft for use in accordance with the methods of the invention is straight along the entire length of the shaft. Alternatively, a portion of the hollow shaft extends linearly along a longitudinal axis and at least one other portion of the shaft extends off the longitudinal axis. For example, the hollow shaft may have a bend in the distal portion of the shaft, a U-shape, or an arcuate or V-shape in at least a portion of the shaft. Examples of such hollow shafts suitable for use with the methods of the invention include but are not limited to the hollow shafts depicted in FIGS. 6A-6C.

Preferably, the methods of the invention are conducted by making an incision in the eye prior to insertion of the deployment device configured to hold the intraocular shunt. Although in particular embodiments, the methods of the invention may be conducted without making an incision in the eye prior to insertion of the deployment device configured to hold the intraocular shunt. In certain embodiments, the distal end of the deployment device (i.e. the portion that is inserted into the eye) has a sharpened point or tip. For example, the distal end of the deployment device includes or is coupled to a needle configured to hold an intraocular shunt. Needles that are configured to hold an intraocular shunt are commercially available from Terumo Medical Corp. (Elkington Md.). In a particular embodiment, the distal end of the deployment device is coupled to a needle having a hollow interior and a beveled tip, and the intraocular shunt is held within the hollow interior of the needle. In another particular embodiment, the distal end of the deployment device is coupled to a needle having a hollow interior and a triple ground point or tip.

The methods of the invention are preferably conducted without needing to remove an anatomical portion or feature of the eye, including but not limited to the trabecular meshwork, the iris, the cornea, or aqueous humor. The methods of the invention are also preferably conducting without inducing substantial ocular inflammation, such as subconjunctival blebbing or endophthalmitis. Such methods are preferably achieved using an ab interno approach by inserting the deployment device comprising the intraocular shunt through the cornea, across the anterior chamber, through the trabecular meshwork and sclera and into a drainage structure such as Schlemm's canal, the subconjunctival space, the episcleral vein, the suprachoroidal space, the intra-Tenon's space or the subarachnoid space. However, the methods of the invention may be conducted using an ab externo approach.

When the methods of the invention are conducted using an ab interno approach, the deployment device is preferably inserted into the eye at an angle above or below the corneal limbus, inserted in contrast with entering through the corneal limbus. Preferably, the deployment device is inserted above the corneal limbus. For example, the deployment device is inserted approximately 0.25 to 3.0 mm, preferably approximately 0.5 to 2.5 mm, more preferably approximately 1.0 mm to 2.0 mm above the corneal limbus, or any specific value within said ranges, e.g., approximately 1.0 mm, approximately 1.1 mm, approximately 1.2 mm, approximately 1.3 mm, approximately 1.4 mm, approximately 1.5 mm, approximately 1.6 mm, approximately 1.7 mm, approximately 1.8 mm, approximately 1.9 mm or approximately 2.0 mm above the corneal limbus.

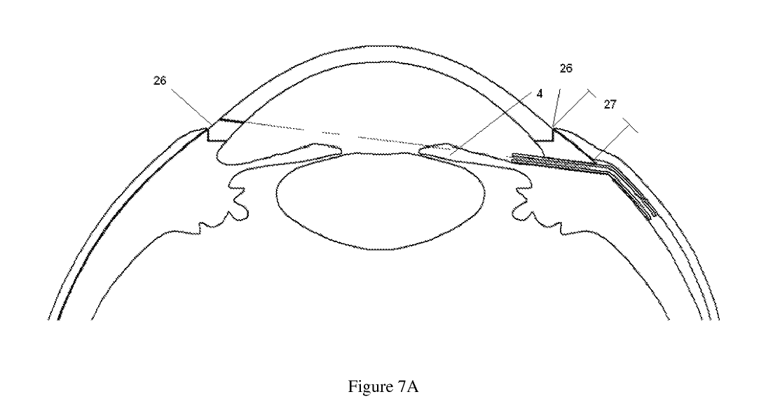

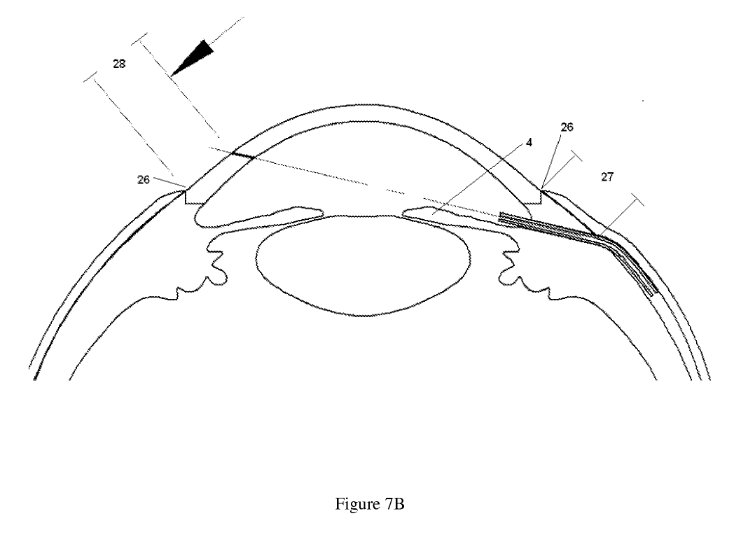

Entering at an angle above or below the corneal limbus is advantageous for placing the shunt farther from the limbus at the exit site. It also adds more distance between the shunt and the iris. FIG. 7 demonstrates the change in location of the shunt sclera exit and the height above the iris in the chamber at different angles of entry using a hollow needle configured to hold an intraocular shunt. As shown in FIG. 7A, needle entry at the limbus 26 results in an exit site distance 27 of approximately 1.6 mm from the limbus 26, and very close proximity to the iris 4. In contrast, a high angle of entry 28 above the limbus 26 (e.g., 2 mm above the limbus 26), results in an exit site distance 27 of approximately 2.1 mm from the limbus 26 and a height well above the iris 4, as shown in FIG. 7B. Without intending to be bound by any theory, placement of the shunt farther from the limbus at the exit site, as provided by an angle of entry above the limbus, is believed to provide access to more lymphatic channels for drainage of aqueous humor, such as the episcleral lymphatic network, in addition to the conjunctival lymphatic system.

Deployment of an intraocular shunt in the eye in accordance with the methods of the invention results in the formation of a passage that directs fluid flow from an area of high pressure in the eye, typically the anterior chamber, to an area of lower pressure within the eye or within the head, to relieve or reduce intraocular pressure. Areas of lower pressure within the eye that are suited for aqueous humor drainage include but are not limited to the intra-Tenon's space, the subconjunctival space, the episceleral vein, the suprachoroidal space and Schlemm's canal. Alternatively, the subarachnoid space may provide a drainage outlet for aqueous humor from the anterior chamber. Preferably, deployment of the shunt results in the formation of a passage for directing fluid flow between the anterior chamber and the intra-Tenon's space.

Deployment of an intraocular shunt such that the inlet (i.e., the portion of the shunt that receives fluid from an anterior chamber of the eye) terminates in the anterior chamber and the outlet (i.e., the portion of the shunt that directs fluid to the intra-Tenon's space) terminates in the intra-Tenon's space provides superior benefits over deployment generally in the subconjunctival space. Deployment of the shunt outlet in the intra-Tenon's space safeguards the integrity of the conjunctiva to allow subconjunctival drainage pathways to successfully form. See, for example, Yu et al., Progress in Retinal and Eye Research, 28: 303-328 (2009)). Additionally, drainage into the intra-Tenon's space provides access to more lymphatic channels than just the conjunctival lymphatic system, such as the episcleral lymphatic network. Moreover, deployment of an intraocular shunt such that the outlet terminates in the intra-Tenon's space avoids having to pierce Tenon's capsule which can otherwise cause complications during glaucoma filtration surgery due to its tough and fibrous nature.

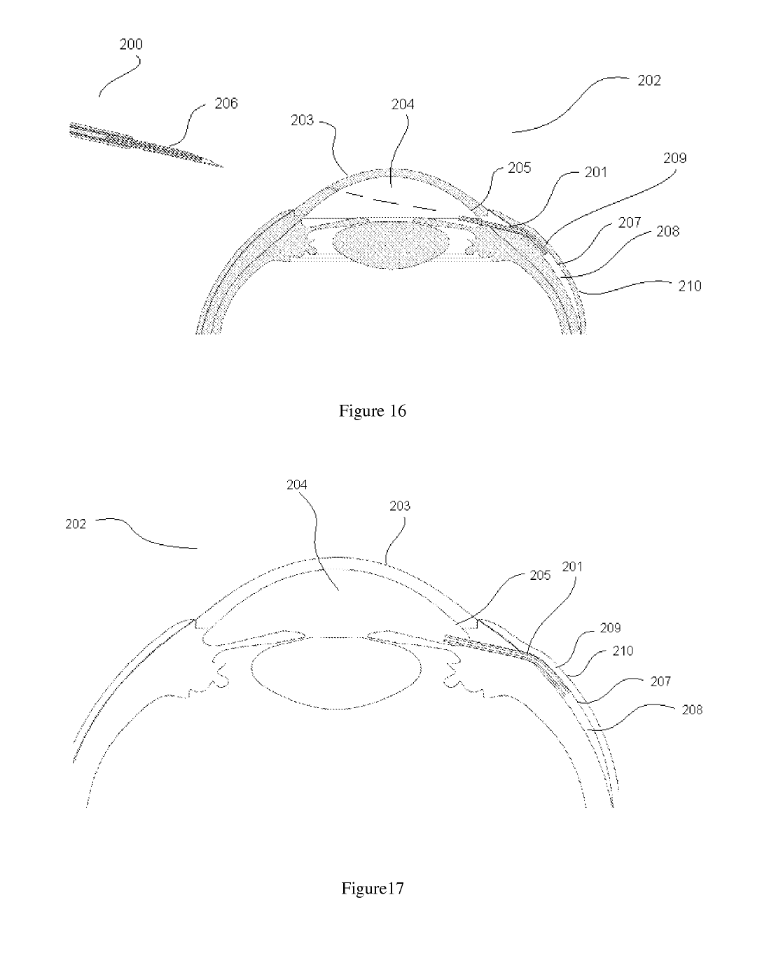

Referring to FIGS. 16 and 17, which show an intraocular shunt placed into the eye such that the shunt forms a passage for fluid drainage from the anterior chamber to the intra-Tenon's space. To place the shunt within the eye, a surgical intervention to implant the shunt is preformed that involves inserting into the eye 202 a deployment device 200 that holds an intraocular shunt 201, and deploying at least a portion of the shunt 201 within intra-Tenon's space 208, within subconjunctival space 209 beneath the conjunctiva 210. In certain embodiments, a hollow shaft 206 of a deployment device 200 holding the shunt 201 enters the eye 202 through the cornea 203 (ab interno approach). The shaft 206 is advanced across the anterior chamber 204 (as depicted by the broken line) in what is referred to as a transpupil implant insertion. The shaft 206 is advanced through the sclera 205 until a distal portion of the shaft 206 is in proximity to Tenon's capsule 207. After piercing the sclera 205 with the hollow shaft 206 of the deployment device 200, resistance to advancement of the shaft 206 encountered by an operator of the deployment device 200 informs the operator that the shaft 206 has contacted Tenon's capsule 207 and is thus in proximity to Tenon's capsule 207.

Numerous techniques may be employed to ensure that after piercing the sclera 205, the hollow shaft 206 does not pierce Tenon's capsule 207. In certain embodiments, the methods of the invention involve the use of a hollow shaft 206, in which a portion of the hollow shaft extends linearly along a longitudinal axis and at least one other portion of the shaft extends off the longitudinal axis. For example, the hollow shaft 206 may have a bend in the distal portion of the shaft, a U-shape, or an arcuate or V-shape in at least a portion of the shaft. Examples of such hollow shafts 206 suitable for use with the methods of the invention include but are not limited to the hollow shafts 206 depicted in FIGS. 6A-6C. In embodiments in which the hollow shaft 206 has a bend at a distal portion of the shaft, intra-Tenon's shunt placement can be achieved by using the bent distal portion of the shaft 206 to push Tenon's capsule 207 away from the sclera 205 without penetrating Tenon's capsule 207. In these embodiments, the tip of the distal end of the shaft 206 does not contact Tenon's capsule 207.

In other embodiments, a straight hollow shaft 206 having a beveled tip is employed. The angle of the beveled tip of the hollow shaft is configured such that after piercing the sclera 205, the hollow shaft 206 does not pierce Tenon's capsule 207. In these embodiments, the shaft 206 is inserted into the eye 202 and through the sclera 205 at an angle such that the bevel of the tip is parallel to Tenon's capsule 207, thereby pushing Tenon's capsule 207 away from the sclera 205, rather than penetrating Tenon's capsule 207, and allowing for deployment of a distal portion of the shunt 201 into the intra-Tenon's space 208.

Once a distal portion of the hollow shaft 206 is within the intra-Tenon's space 208, at least a portion of the device is rotated, thereby reducing the friction between the portion of the device that is in contact with the scleral tissue and the scleral tissue itself. Reduction in friction allows for deployment of the shunt from the device and then removal of the device from the eye without disturbing the tissue of the eye. After rotating the device, the shunt 201 is then deployed from the shaft 206 of the deployment device 200, producing a conduit between the anterior chamber 204 and the intra-Tenon's space 208 to allow aqueous humor to drain from the anterior chamber 204 (See FIGS. 16 and 17).

In another embodiment, the methods of the invention further involves injecting an aqueous solution into the eye below Tenon's capsule in order to balloon the capsule away from the sclera. The increase in intra-Tenon's space caused by the ballooning of Tenon's capsule is helpful for positioning of the outlet of the shunt in the intra-Tenon's space. The solution is injected prior to the shaft piercing the sclera and entering the intra-Tenon's space. Suitable aqueous solutions include but are not limited to Dulbecco's Phosphate Buffered Saline (DPBS), Hank's Balanced Salt Solution (HBSS), Phosphate-Buffered Saline (PBS), Earle's Balanced Salt Solution (EBSS), or other balanced salt solutions known in the art. In some embodiments, the methods of the invention involve injecting a viscoelastic fluid into the eye. Preferably, the methods of the invention are conducted without the use of a viscoelastic fluid. The methods of the invention can be conducted using any shunt deployment device known in the art. Examples of deployment devices that are suitable for use with the methods of the invention include but are not limited to the devices described in U.S. Pat. No. 6,007,511, U.S. Pat. No. 6,544,249, and U.S. Publication No. US2008/0108933, the contents of each of which are hereby incorporated by reference in their entireties.

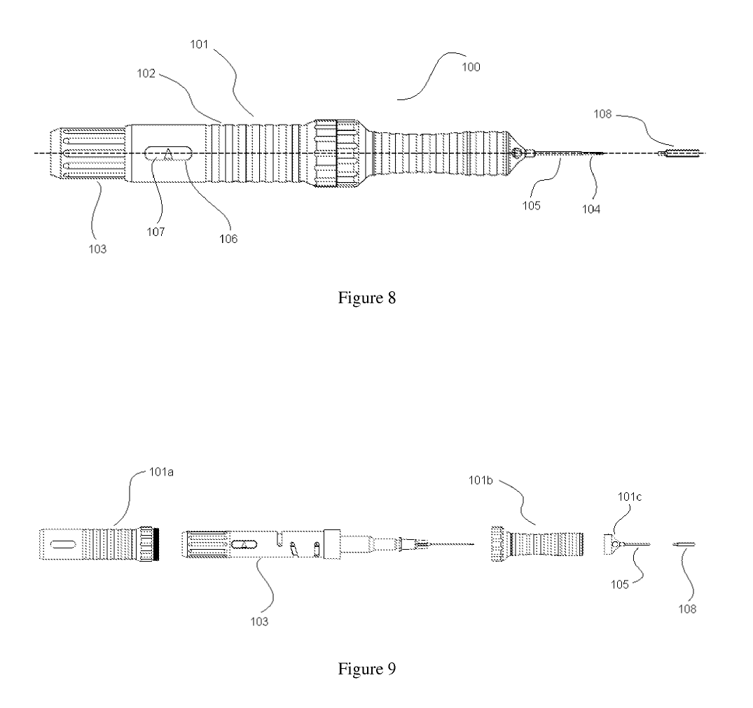

In other embodiments, the methods of the invention are conducted using the deployment device 100 depicted in FIG. 8. While FIG. 8 shows a handheld manually operated shunt deployment device, it will be appreciated that devices of the invention may be coupled with robotic systems and may be completely or partially automated. As shown in FIG. 8, deployment device 100 includes a generally cylindrical body or housing 101, however, the body shape of housing 101 could be other than cylindrical. Housing 101 may have an ergonomical shape, allowing for comfortable grasping by an operator. Housing 101 is shown with optional grooves 102 to allow for easier gripping by a surgeon.

Housing 101 is shown having a larger proximal portion that tapers to a distal portion. The distal portion includes a hollow sleeve 105. The hollow sleeve 105 is configured for insertion into an eye and to extend into an anterior chamber of an eye. The hollow sleeve is visible within an anterior chamber of an eye. The sleeve may include an edge at a distal end that provides resistance feedback to an operator upon insertion of the deployment device 100 within an eye of a person. Upon advancement of the device 100 across an anterior chamber of the eye, the hollow sleeve 105 will eventually contact the sclera, providing resistance feedback to an operator that no further advancement of the device 100 is necessary. The edge of the sleeve 105, prevents the shaft 104 from accidentally being pushed too far through the sclera. A temporary guard 108 is configured to fit around sleeve 105 and extend beyond an end of sleeve 105. The guard is used during shipping of the device and protects an operator from a distal end of a hollow shaft 104 that extends beyond the end of the sleeve 105. The guard is removed prior to use of the device.

Housing 101 is open at its proximal end, such that a portion of a deployment mechanism 103 may extend from the proximal end of the housing 101. A distal end of housing 101 is also open such that at least a portion of a hollow shaft 104 may extend through and beyond the distal end of the housing 101. Housing 101 further includes a slot 106 through which an operator, such as a surgeon, using the device 100 may view an indicator 107 on the deployment mechanism 103.

Housing 101 may be made of any material that is suitable for use in medical devices. For example, housing 101 may be made of a lightweight aluminum or a biocompatible plastic material. Examples of such suitable plastic materials include polycarbonate and other polymeric resins such as DELRIN and ULTEM. In certain embodiments, housing 101 is made of a material that may be autoclaved, and thus allow for housing 101 to be re-usable. Alternatively, device 100, may be sold as a one-time-use device, and thus the material of the housing does not need to be a material that is autoclavable.

Housing 101 may be made of multiple components that connect together to form the housing. FIG. 9 shows an exploded view of deployment device 100. In this figure, housing 101, is shown having three components 101a, 101b, and 101c. The components are designed to screw together to form housing 101. FIG. 9 also shows deployment mechanism 103. The housing 101 is designed such that deployment mechanism 103 fits within assembled housing 101. Housing 101 is designed such that components of deployment mechanism 103 are movable within housing 101.

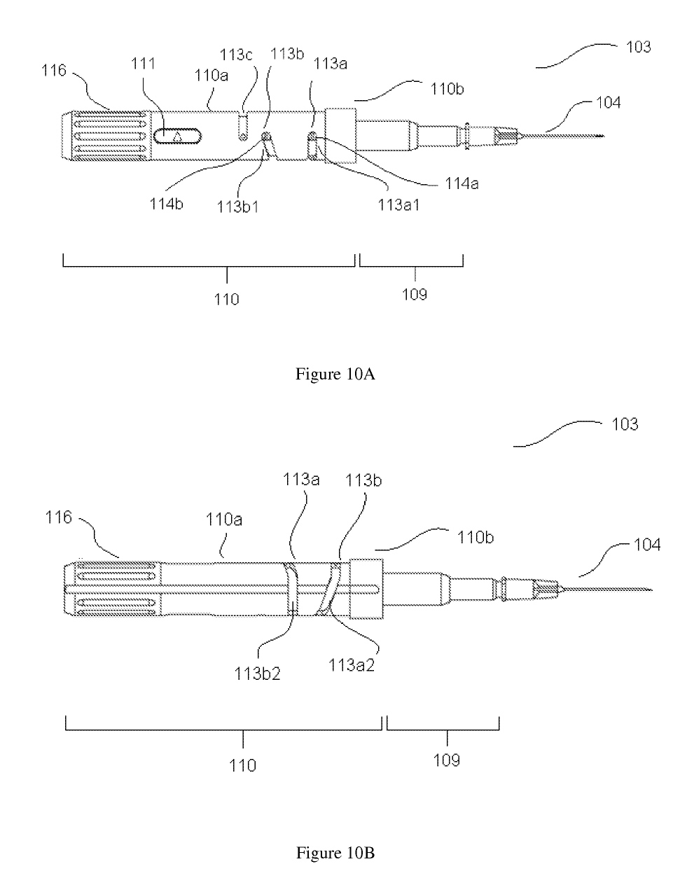

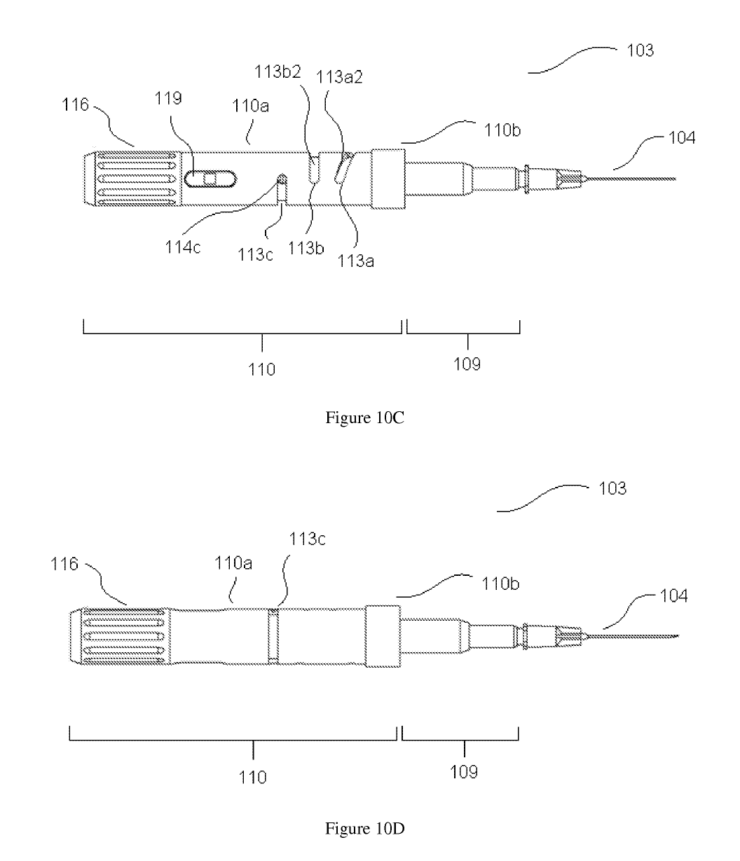

FIGS. 10A to 10D show different enlarged views of the deployment mechanism 103. Deployment mechanism 103 may be made of any material that is suitable for use in medical devices. For example, deployment mechanism 103 may be made of a lightweight aluminum or a biocompatible plastic material. Examples of such suitable plastic materials include polycarbonate and other polymeric resins such as DELRIN and ULTEM. In certain embodiments, deployment mechanism 103 is made of a material that may be autoclaved, and thus allow for deployment mechanism 103 to be re-usable. Alternatively, device 100 may be sold as a one-time-use device, and thus the material of the deployment mechanism does not need to be a material that is autoclavable.

Deployment mechanism 103 includes a proximal portion 109 and a distal portion 110. The deployment mechanism 103 is configured such that proximal portion 109 is movable within distal portion 110. More particularly, proximal portion 109 is capable of partially retracting to within distal portion 110.

In this embodiment, the proximal portion 109 is shown to taper to a connection with a hollow shaft 104. This embodiment is illustrated such that the connection between the hollow shaft 104 and the proximal portion 109 of the deployment mechanism 103 occurs inside the housing 101. In other embodiments, the connection between hollow shaft 104 and the proximal portion 109 of the deployment mechanism 103 may occur outside of the housing 101. Hollow shaft 104 may be removable from the proximal portion 109 of the deployment mechanism 103. Alternatively, the hollow shaft 104 may be permanently coupled to the proximal portion 109 of the deployment mechanism 103.

Generally, hollow shaft 104 is configured to hold an intraocular shunt, such as the intraocular shunts according to the invention. The shaft 104 may be any length. A usable length of the shaft may be anywhere from about 5 mm to about 40 mm, and is 15 mm in certain embodiments. In certain embodiments, the shaft is straight. In other embodiments, shaft is of a shape other than straight, for example a shaft having a bend along its length or a shaft as depicted in FIGS. 6A-6C.

A distal portion of the deployment mechanism includes optional grooves 116 to allow for easier gripping by an operator for easier rotation of the deployment mechanism, which will be discussed in more detail below. The distal portion 110 of the deployment mechanism also includes at least one indicator that provides feedback to an operator as to the state of the deployment mechanism. The indicator may be any type of indicator know in the art, for example a visual indicator, an audio indicator, or a tactile indicator. FIG. 10 shows a deployment mechanism having two indicators, a ready indicator 111 and a deployed indicator 119. Ready indicator 111 provides feedback to an operator that the deployment mechanism is in a configuration for deployment of an intraocular shunt from the deployment device 100. The indicator 111 is shown in this embodiment as a green oval having a triangle within the oval. Deployed indicator 119 provides feedback to the operator that the deployment mechanism has been fully engaged and has deployed the shunt from the deployment device 100. The deployed indicator 119 is shown in this embodiment as a yellow oval having a black square within the oval. The indicators are located on the deployment mechanism such that when assembled, the indicators 111 and 119 may be seen through slot 106 in housing 101.

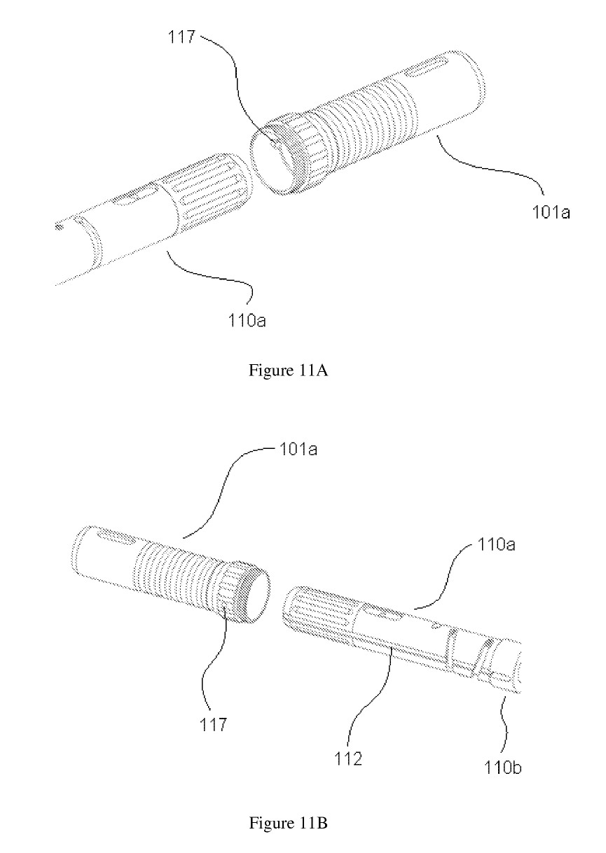

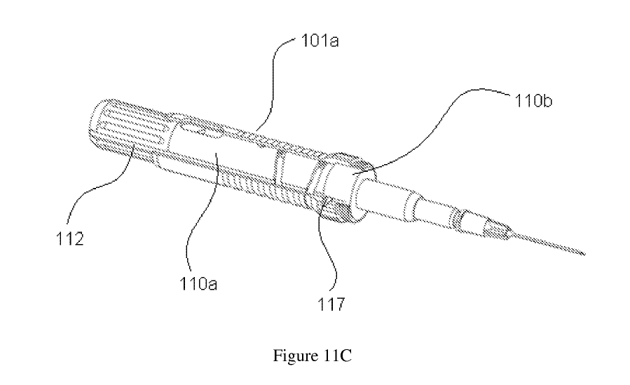

The distal portion 110 includes a stationary portion 110b and a rotating portion 110a. The distal portion 110 includes a channel 112 that runs part of the length of stationary portion 110b and the entire length of rotating portion 110a. The channel 112 is configured to interact with a protrusion 117 on an interior portion of housing component 101a (FIGS. 11A and 11B). During assembly, the protrusion 117 on housing component 101a is aligned with channel 112 on the stationary portion 110b and rotating portion 110a of the deployment mechanism 103. The distal portion 110 of deployment mechanism 103 is slid within housing component 101a until the protrusion 117 sits within stationary portion 110b (FIG. 11C). Assembled, the protrusion 117 interacts with the stationary portion 110b of the deployment mechanism 103 and prevents rotation of stationary portion 110b. In this configuration, rotating portion 110a is free to rotate within housing component 101a.

Referring back to FIG. 10, the rotating portion 110a of distal portion 110 of deployment mechanism 103 also includes channels 113a, 113b, and 113c. Channel 113a includes a first portion 113a1 that is straight and runs perpendicular to the length of the rotating portion 110a, and a second portion 113a2 that runs diagonally along the length of rotating portion 110a, downwardly toward a distal end of the deployment mechanism 103. Channel 113b includes a first portion 113b1 that runs diagonally along the length of the rotating portion 110a, upwardly toward a proximal end of the deployment mechanism 103, and a second portion that is straight and runs perpendicular to the length of the rotating portion 110a. The point at which first portion 113a1 transitions to second portion 113a2 along channel 113a, is the same as the point at which first portion 113b1 transitions to second portion 113b2 along channel 113b. Channel 113c is straight and runs perpendicular to the length of the rotating portion 110a. Within each of channels 113a, 113b, and 113c, sit members 114a, 114b, and 114c respectively. Members 114a, 114b, and 114c are movable within channels 113a, 113b, and 113c. Members 114a, 114b, and 114c also act as stoppers that limit movement of rotating portion 110a, which thereby limits axial movement of the shaft 104.

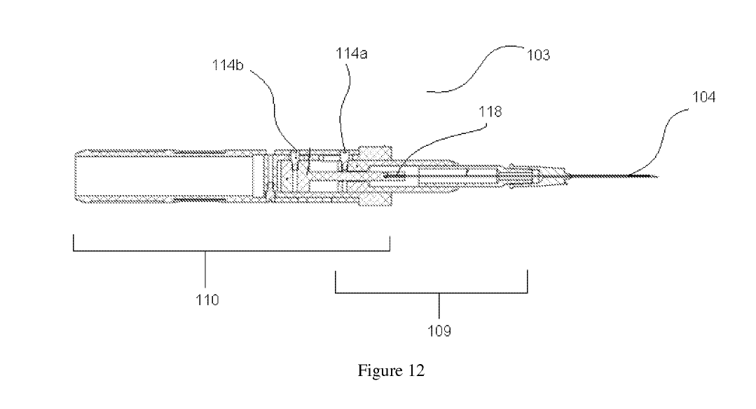

FIG. 12 shows a cross-sectional view of deployment mechanism 103. Member 114a is connected to the proximal portion 109 of the deployment mechanism 103. Movement of member 114a results in retraction of the proximal portion 109 of the deployment mechanism 103 to within the distal portion 110 of the deployment mechanism 103. Member 114b is connected to a pusher component 118. The pusher component 118 extends through the proximal portion 109 of the deployment mechanism 103 and extends into a portion of hollow shaft 104. The pusher component is involved in deployment of a shunt from the hollow shaft 104. An exemplary pusher component is a plunger. Movement of member 114b engages pusher 118 and results in pusher 118 advancing within hollow shaft 104.

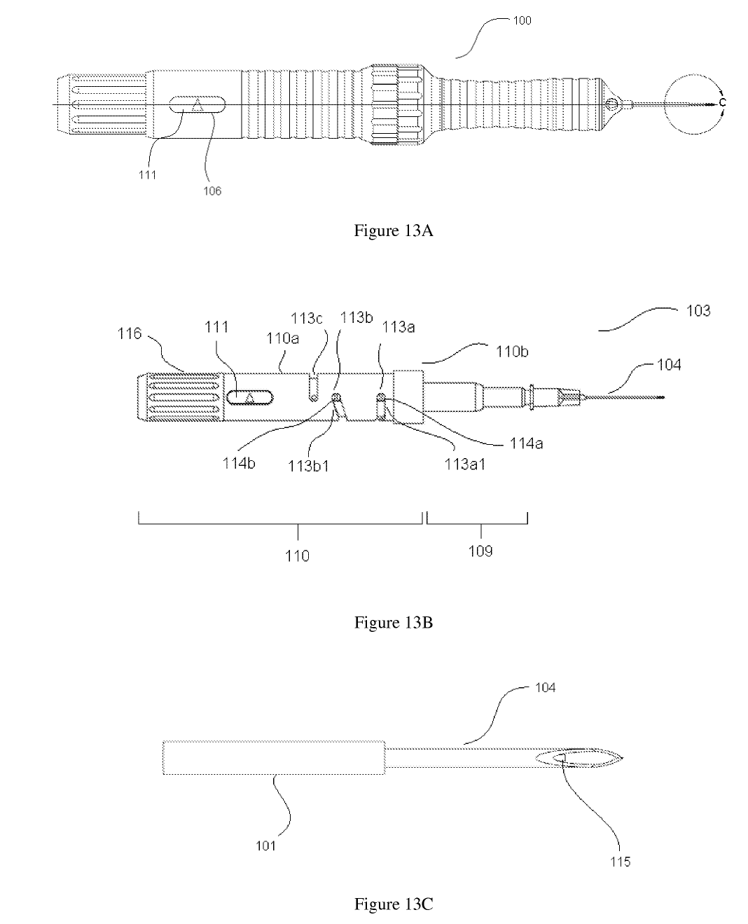

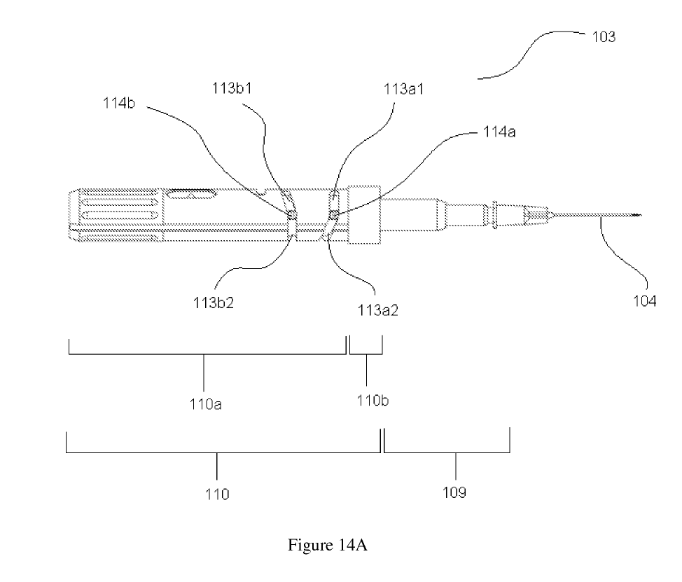

Reference is now made to FIGS. 13-15, which accompany the following discussion regarding deployment of a shunt 115 from deployment device 100. FIG. 13A shows deployment device 100 is a pre-deployment configuration. In this configuration, shunt 115 is loaded within hollow shaft 104 (FIG. 13C). As shown in FIG. 13C, shunt 115 is only partially within shaft 104, such that a portion of the shunt is exposed. However, the shunt 115 does not extend beyond the end of the shaft 104. In other embodiments, the shunt 115 is completely disposed within hollow shaft 104. The shunt 115 is loaded into hollow shaft 104 such that the shunt abuts pusher component 118 within hollow shaft 104. A distal end of shaft 104 is beveled to assist in piercing tissue of the eye.

Additionally, in the pre-deployment configuration, a portion of the shaft 104 extends beyond the housing 101 (FIG. 13C). The deployment mechanism is configured such that member 114a abuts a proximal end of the first portion 113a1 of channel 113a, and member 114b abut a proximal end of the first portion 113b1 of channel 113b (FIG. 13B). In this configuration, the ready indicator 111 is visible through slot 106 of the housing 101, providing feedback to an operator that the deployment mechanism is in a configuration for deployment of an intraocular shunt from the deployment device 100 (FIG. 13A). In this configuration, the device 100 is ready for insertion into an eye (insertion configuration or pre-deployment configuration). Methods for inserting and implanting shunts are discussed in further detail below.

Once the device has been inserted into the eye and advanced to a location to where the shunt will be deployed, the shunt 115 may be deployed from the device 100. The deployment mechanism 103 is a two-stage system. The first stage is engagement of the pusher component 118 and the second stage is retraction of the proximal portion 109 to within the distal portion 110 of the deployment mechanism 103. Rotation of the rotating portion 110a of the distal portion 110 of the deployment mechanism 103 sequentially engages the pusher component and then the retraction component. It should be noted that rotating portion 110a is distinct from the portion of the deployment device that is rotated to loosen the portion of the deployment device that is inserted into the eye from the surrounding eye tissue, as described herein.

In the first stage of shunt deployment, the pusher component is engaged and the pusher partially deploys the shunt from the deployment device. During the first stage, rotating portion 110a of the distal portion 110 of the deployment mechanism 103 is rotated, resulting in movement of members 114a and 114b along first portions 113a1 and 113b1 in channels 113a and 113b. Since the first portion 113a1 of channel 113a is straight and runs perpendicular to the length of the rotating portion 110a, rotation of rotating portion 110a does not cause axial movement of member 114a. Without axial movement of member 114a, there is no retraction of the proximal portion 109 to within the distal portion 110 of the deployment mechanism 103. Since the first portion 113b1 of channel 113b runs diagonally along the length of the rotating portion 110a, upwardly toward a proximal end of the deployment mechanism 103, rotation of rotating portion 110a causes axial movement of member 114b toward a proximal end of the device. Axial movement of member 114b toward a proximal end of the device results in forward advancement of the pusher component 118 within the hollow shaft 104. Such movement of pusher component 118 results in partially deployment of the shunt 115 from the shaft 104.

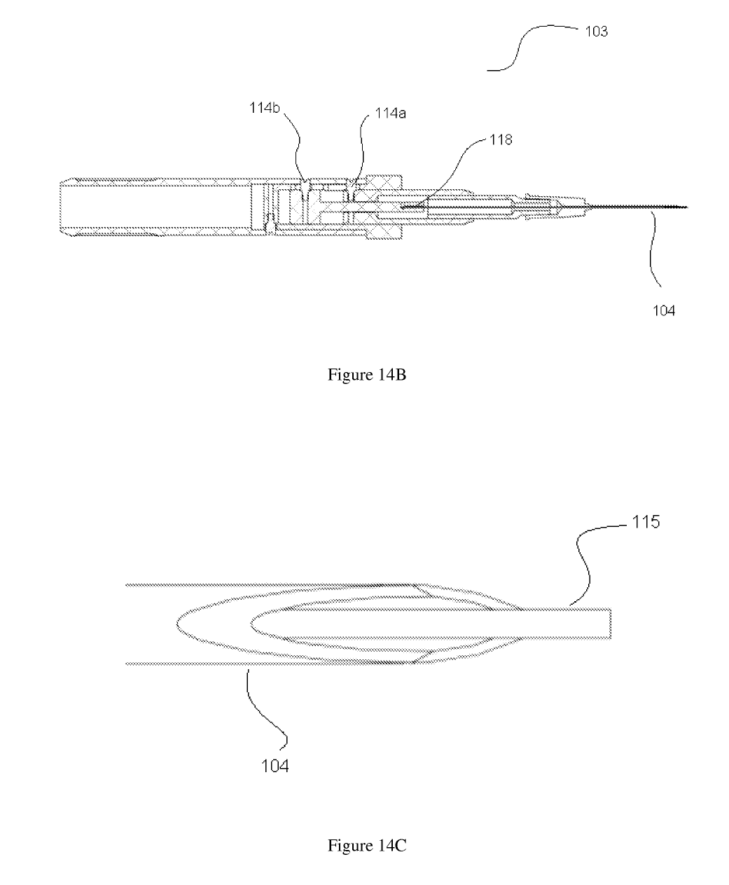

FIGS. 14A to 14C show schematics of the deployment mechanism at the end of the first stage of deployment of the shunt from the deployment device. As is shown FIG. 14A, members 114a and 114b have finished traversing along first portions 113a1 and 113b1 of channels 113a and 113b. Additionally, pusher component 118 has advanced within hollow shaft 104 (FIG. 14B), and shunt 115 has been partially deployed from the hollow shaft 104 (FIG. 14C). As is shown in these figures, a portion of the shunt 115 extends beyond an end of the shaft 104.

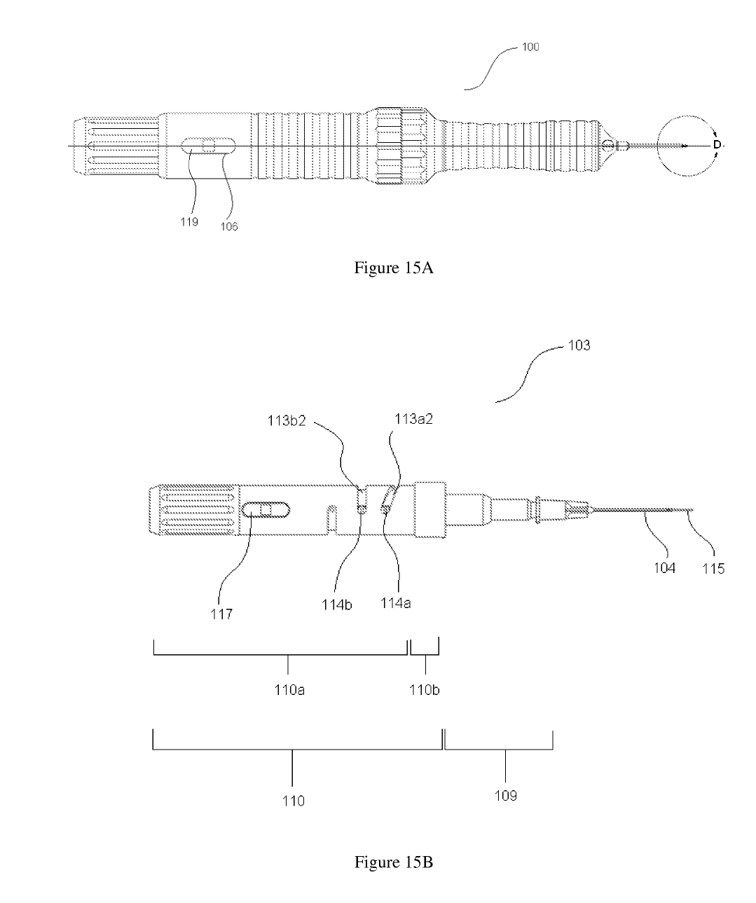

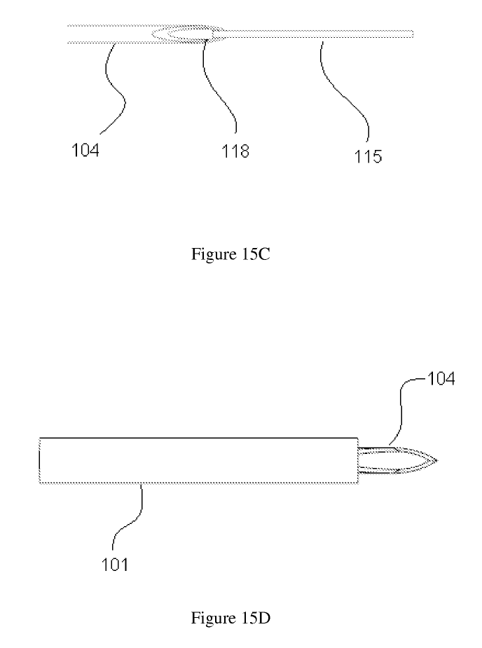

In the second stage of shunt deployment, the retraction component is engaged and the proximal portion of the deployment mechanism is retracted to within the distal portion of the deployment mechanism, thereby completing deployment of the shunt from the deployment device. During the second stage, rotating portion 110a of the distal portion 110 of the deployment mechanism 103 is further rotated, resulting in movement of members 114a and 114b along second portions 113a2 and 113b2 in channels 113a and 113b. Since the second portion 113b2 of channel 113b is straight and runs perpendicular to the length of the rotating portion 110a, rotation of rotating portion 110a does not cause axial movement of member 114b. Without axial movement of member 114b, there is no further advancement of pusher 112. Since the second portion 113a2 of channel 113a runs diagonally along the length of the rotating portion 110a, downwardly toward a distal end of the deployment mechanism 103, rotation of rotating portion 110a causes axial movement of member 114a toward a distal end of the device. Axial movement of member 114a toward a distal end of the device results in retraction of the proximal portion 109 to within the distal portion 110 of the deployment mechanism 103. Retraction of the proximal portion 109, results in retraction of the hollow shaft 104. Since the shunt 115 abuts the pusher component 118, the shunt remains stationary at the hollow shaft 104 retracts from around the shunt 115 (FIG. 15C). The shaft 104, retracts almost completely to within the housing 101. During both stages of the deployment process, the housing 101 remains stationary and in a fixed position.

FIG. 15A shows a schematic of the device 100 after deployment of the shunt 115 from the device 100. FIG. 15B shows a schematic of the deployment mechanism at the end of the second stage of deployment of the shunt from the deployment device. As is shown in FIG. 15B, members 114a and 114b have finished traversing along second portions 113a1 and 113b1 of channels 113a and 113b. Additionally, proximal portion 109 has retracted to within distal portion 110, thus resulting in retraction of the hollow shaft 104 to within the housing 101. FIG. 15D shows an enlarged view of the distal portion of the deployment device after deployment of the shunt. This figure shows that the hollow shaft 104 is not fully retracted to within the housing 101 of the deployment device 100. However, in certain embodiments, the shaft 104 may completely retract to within the housing 101.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

What is claimed is:

1. A method of implanting an intraocular shunt into an eye, the method comprising the steps of:
inserting into the eye a portion of a deployment device comprising an intraocular shunt;
loosening the portion of the deployment device from eye tissue surrounding the portion of the deployment device; and
deploying the shunt into the eye from the deployment device.

2. The method of claim 1, wherein deployment of the shunt results in the formation of a passage that directs fluid flow from an area of high pressure to an area of lower pressure within the eye.

3. The method of claim 2, wherein the area of high pressure is the anterior chamber of the eye.

4. The method of claim 2, wherein the area of lower pressure is selected from the group consisting of: the intra-Tenon's space; the subconjunctival space; the episcleral vein; the suprachoroidal space; and Schlemm's canal.

5. The method of claim 4, wherein the area of lower pressure is the intra-Tenon's space.

6. The method of claim 1, wherein said loosening step comprises rotating at least a portion of the deployment device.

7. The method of claim 6, wherein a proximal portion of the deployment device is rotated.

8. The method of claim 6, wherein a distal portion of the deployment device is rotated.

9. The method of claim 6, wherein the entire deployment device is rotated.

10. The method of claim 1, wherein the inserting step comprises ab interno insertion of the deployment device into the eye.

11. The method of claim 10, wherein ab interno insertion comprises inserting the deployment device into the eye above the corneal limbus.

12. The method of claim 10, wherein ab interno insertion comprises inserting the deployment device into the eye below the corneal limbus

13. The method of claim 1, wherein the deployment device is inserted into the eye without removing an anatomical feature of the eye.

14. The method of claim 13, wherein the anatomical feature is selected from the group consisting of: the trabecular meshwork, the iris, the cornea, and the aqueous humor.

15. The method of claim 1, wherein the method is performed without inducing subconunctival blebbing or endophthalmitis.

16. The method of claim 1, wherein the eye tissue surrounding the portion of the deployment device is scleral tissue.

17. A method of implanting an intraocular shunt into the eye, the method comprising the steps of:
inserting into the eye a portion of a deployment device comprising an intraocular shunt without removing an anatomical feature of the eye;
loosening the portion of the deployment device from eye tissue surrounding the portion of the deployment device; and
deploying the shunt into the eye from the deployment device.

18. The method of claim 17, wherein the anatomical feature is selected from the group consisting of: the trabecular meshwork or a portion thereof, the iris, the cornea, and the aqueous humor.

19. The method of claim 17, wherein the method is performed without inducing subconjunctival blebbing or endophthalmitis.

20. The method of claim 17, wherein deployment of the shunt results in the formation of a passage that directs fluid flow from an area of high pressure to an area of lower pressure within the eye.

21. The method of claim 20, wherein the area of high pressure is the anterior chamber of the eye.

22. The method of claim 20, wherein the area of lower pressure is selected from the group consisting of: the intra-Tenon's space; the subconjunctival space; the episcleral vein; the suprachoroidal space; and Schlemm's canal.

23. The method of claim 22, wherein the area of lower pressure is the intra-Tenon's space.

24. The method of claim 17, wherein said loosening step comprises rotating at least a portion of the deployment device.

25. The method of claim 24, wherein a proximal portion of the deployment device is rotated.

26. The method of claim 24, wherein a distal portion of the deployment device is rotated.

27. The method of claim 24, wherein the entire deployment device is rotated.

28. The method of claim 17, wherein the eye tissue surrounding the portion of the deployment device is scleral tissue.

29. A method for implanting an intraocular shunt within an eye, the method comprising the step of:
inserting into the eye above the corneal limbus a portion of a deployment device comprising an intraocular shunt, and
deploying the shunt into the eye.

30. The method of claim 29, wherein the deployment device is inserted into the eye at least 1 mm above the corneal limbus.

31. The method of claim 29, wherein the deployment device is inserted into the eye at least 2 mm above the corneal limbus.

32. The method of claim 29, wherein deployment of the shunt results in the formation of a passage that directs fluid flow from an area of high pressure to an area of lower pressure within the eye.

33. The method of claim 32, wherein the area of high pressure is the anterior chamber of the eye.

34. The method of claim 32, wherein the area of lower pressure is selected from the group consisting of: the intra-Tenon's space; the subconjunctival space; the episcleral vein; the suprachoroidal space; and Schlemm's canal.

35. The method of claim 34, wherein the area of lower pressure is the intra-Tenon's space.

36. The method of claim 39, wherein the deployment device is inserted into the eye without removing an anatomical feature of the eye.

37. The method of claim 36, wherein the anatomical feature is selected from the group consisting of: the trabecular meshwork, the iris, the cornea, and the aqueous humor.

38. The method of claim 29, wherein the method is performed without inducing subconjunctival blebbing, or endophthalmitis.
